(19) United States
(12) Patent Application Publication
(10) Pub. No.: US 2017/0274013 A1
(43) Pub. Date: Sep. 28, 2017

(54) WOUND FLUID ELEVATED PROTEASE ENZYME INHIBITION THROUGH CAMELID BLOOD PRODUCTS

(71) Applicant: Patrick T. Prendergast, New South Wales (AU)

(72) Inventor: Patrick T. Prendergast, New South Wales (AU)

(21) Appl. No.: 15/510,969

(22) PCT Filed: Sep. 15, 2014
(86) PCT No.: PCT/IE2014/000014

§ 371 Date: Mar. 13, 2017

(30) Foreign Application Priority Data
Sep. 16, 2013 (IE) 2013/0316

(51) Int. Cl. A61K 35/16; A61K 9/06; C07K 16/06; C07K 16/40; A61K 38/57; C07K 14/81; A61K 9/19; A61K 9/00

(52) U.S. Cl. CPC A61K 35/16; A61K 9/19; A61K 9/06; A61K 9/0014; C07K 16/40; A61K 38/57; C07K 14/81; C07K 16/065; C07K 2317/22; C07K 2317/76

(57) ABSTRACT

Camelid blood products their peptide isolates and synthetic sequences for wound fluid elevated protease enzyme inhibition. The present invention provides evidence that Metalloprotease and other Protease enzyme (e.g. elastase) peptide inhibitors are present in camelid serum/plasma can be used alone or combined with other agents to enhance healing in the treatment of chronic wounds and burns by inhibiting elevated wound fluid protease activity. These protease enzymes inhibitors present in camelid serum/plasma can be demonstrated to inhibit chronic non-healing wound fluid proteolytic enzymes by the use of wound fluid assay kits specifically designed to measure wound fluid protease activity. The serum/plasma and isolated or synthesised peptides of this patent have use in the treatment of a range of cosmetic skin indications and diseases such as disorders of the gastrointestinal tract, cardiovascular conditions and specifically in the treatment of wound healing and burns and in scar tissue healing.

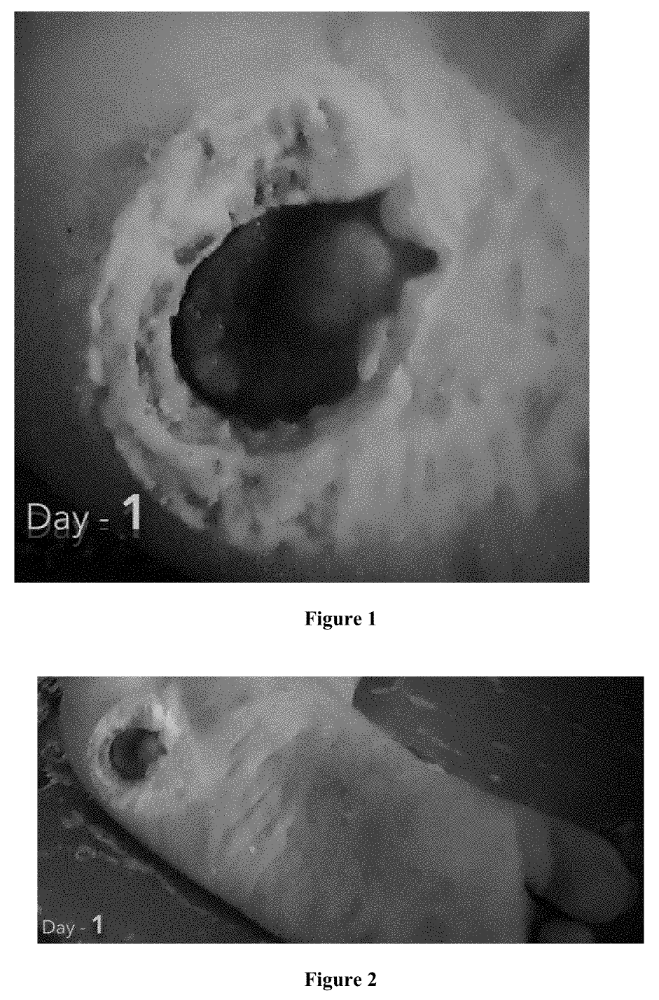

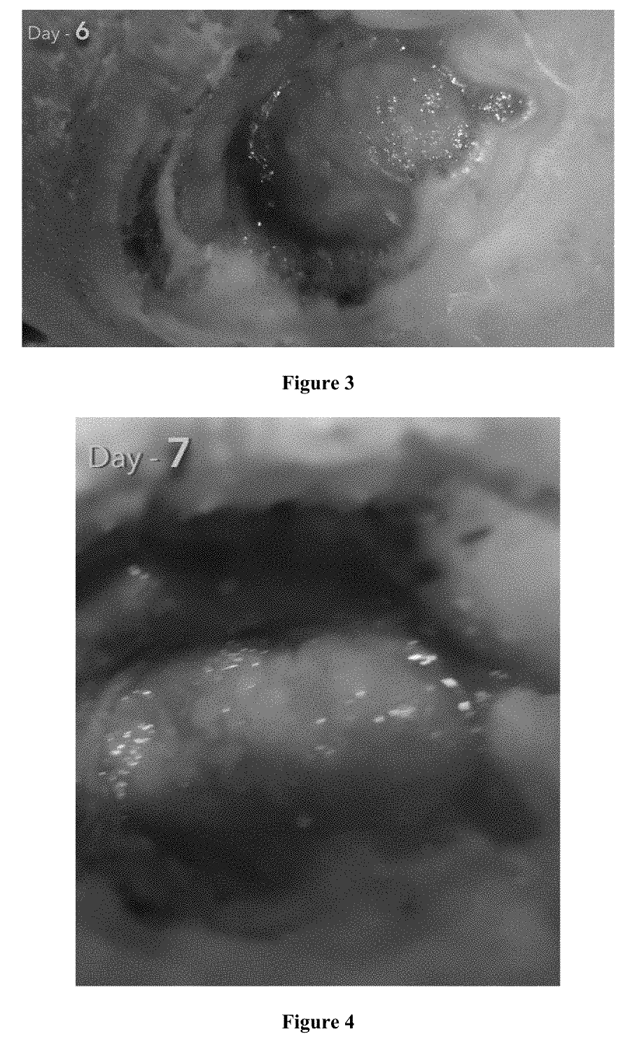

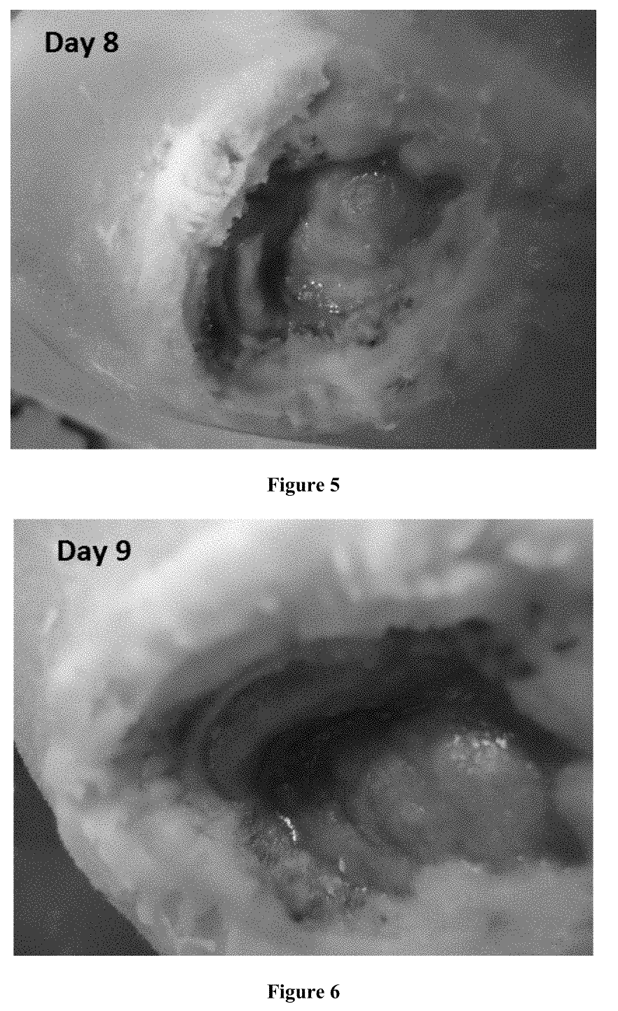

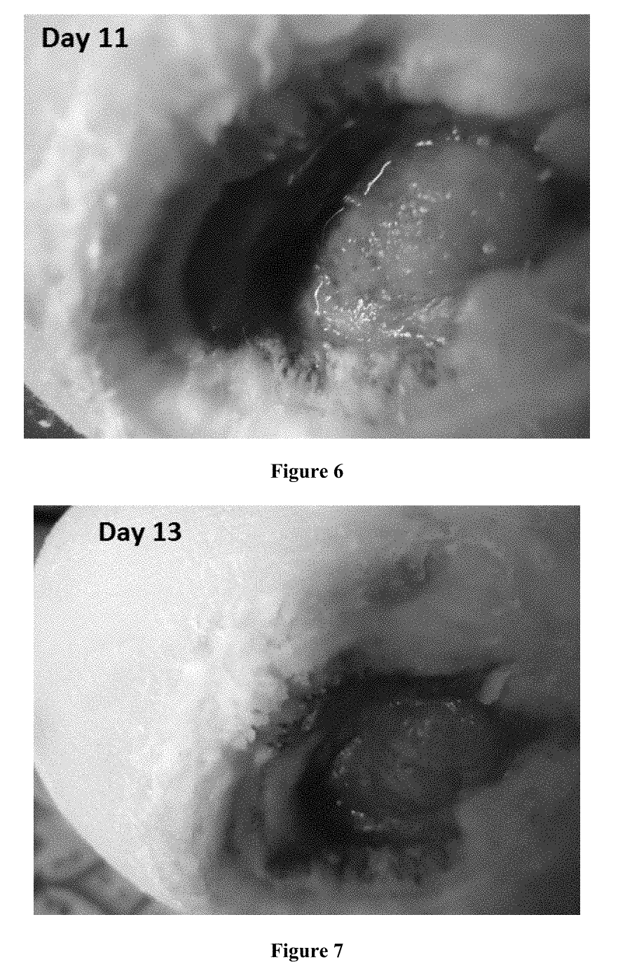

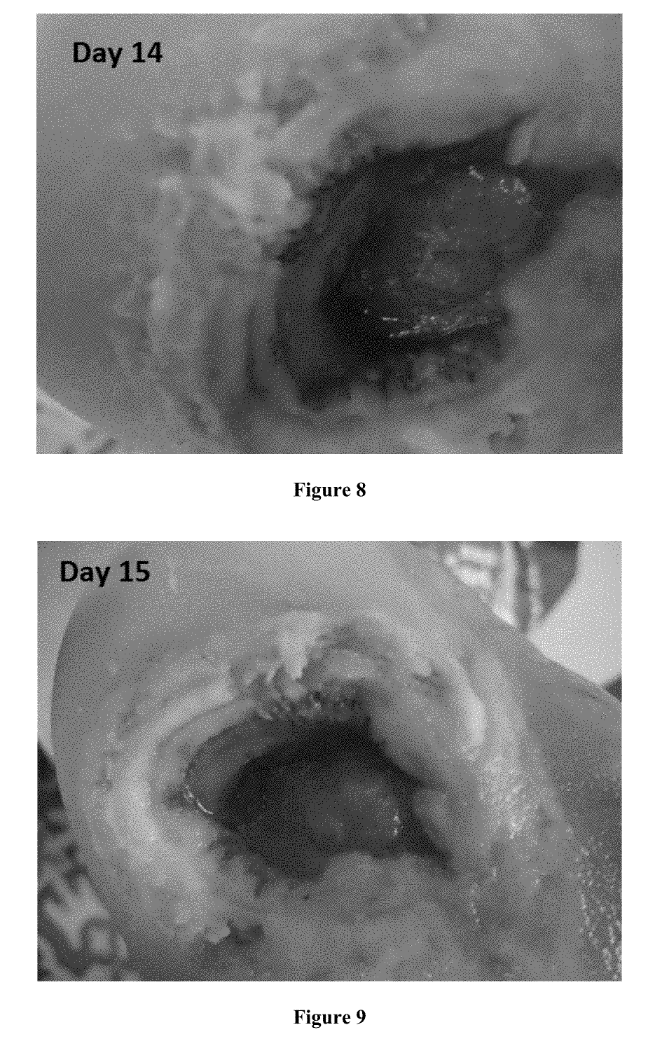

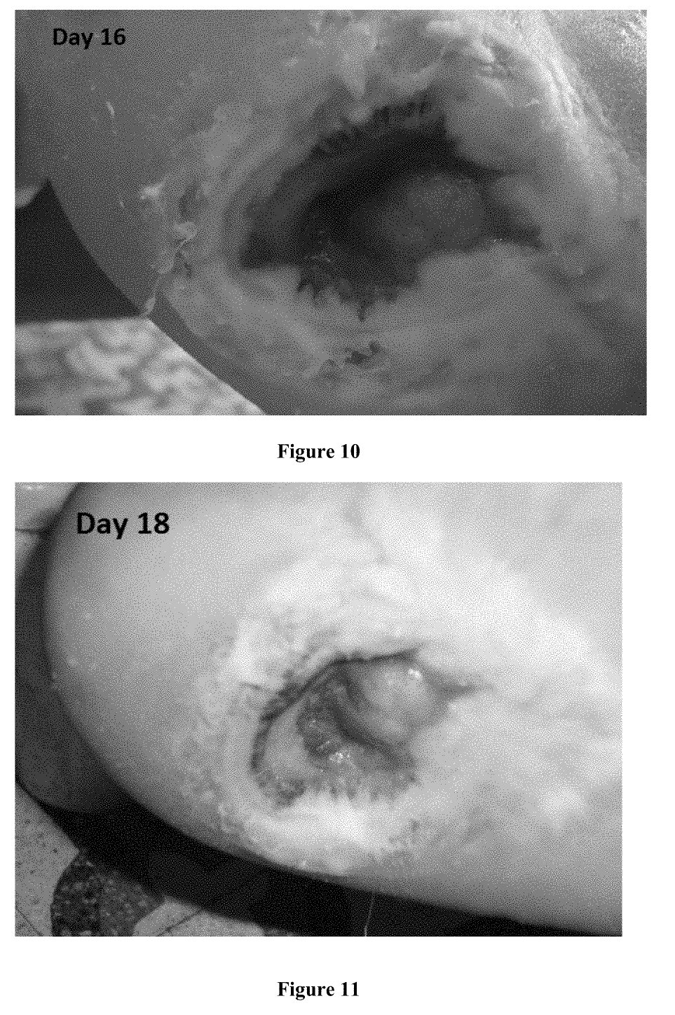

### FIELD OF THE INVENTION

The present invention relates to protease/metalloprotease enzyme inhibitory peptides discovered to be present in camelid serum/plasma or isolated from same. More specifically these enzyme inhibitory peptides isolated from camelid blood are active against human metalloproteinases and other human proteases enzymes and are effective in the treatment of chronic wounds and disorders of the gastrointestinal tract. These protease/metalloprotease inhibitory peptides are naturally present in camelid blood and are not present in such elevated levels or with such broad spectrum inhibition in other ruminants and can be isolated from same for use in therapeutic formulations for wound healing and gastrointestinal tract conditions which require inhibition of elevated tissue protease/metalloprotease enzymes.

### BACKGROUND OF THE INVENTION

Proteinases (metalloprotease) are naturally occurring enzymes present in many tissues of the body. These enzymes act to degrade proteins, normally in a controlled and specific manner. To prevent the uncontrolled destruction of target proteins the activity of these proteolytic enzymes are modulated by inhibitor serum peptides. Thus, the combined and balanced actions of proteinases and inhibitors act to control the level of biologically active or structurally important proteins of the body, thereby regulating many important physiological processes.

One important group of proteinases are the metalloproteinases. These enzymes are characterised by their requirement for the presence of a metal ion in order to catalyse proteolysis. Approximately 17 different metalloproteinases have been identified and/or cloned which share significant sequence homology. The metalloproteinase family can be subdivided into five groups according to their structural and functional properties: (i) the collagenases (metalloproteinases-1, 8 and 13); (ii) the gelatinases A and B (metalloproteinase-2 and metalloproteinase-9) (iii) the stromelysins 1 and 2 (metalloproteinases-3 and 10); (iv) matrilysin (MMP-7); enamelysin (MMP-20), macrophage metalloelastase (MMP-12), and MMP-19 (making up the classical metalloproteinases): (v) the membrane-type metalloproteinases (MT-MMP-1 to 4 and stromelysin-3, MMP-11) (W. Bode et al., Ann N.Y. Acad Sci. 878, 73, 1999). These metalloproteinases share a common multi-domain structure, but are glycosylated to different extents and at different sites. According to sequence alignment, the assembly of these domains might have been an early evolutionary event, followed by diversification. Collectively, metalloproteinases can degrade all the major components of the extracellular matrix (ECM).

The homeostasis of the ECM is controlled by a delicate balance between the synthesis of ECM proteins, production of ECM-degrading extracellular matrix metalloproteinases (MMPs), and the presence of metalloproteinase inhibitors.

One family of metalloproteinases inhibitor peptides are the tissue inhibitors of metalloproteinases (TIMPs). The TIMP family is comprised of at least four distinct members (TIMP-1 to 4) which possess 12 conserved, cysteine residues and express metalloproteinase inhibitory activity by forming non-covalent complexes with metalloproteinases enzymes. Specifically TIMPs bind to the highly conserved active zinc-binding site of the metalloproteinases in a 1:1 stoichiometry, but can also bind at other domains of metalloproteinase-2 and metalloproteinase-9.

Besides their inhibitory role. TIMPs have been identified in a diverse range of biological tissues such as bone, amniotic fluid, cartilage, aortic endothelial cells and skin fibroblasts of humans. These tissues require substantial purification in order to isolate metalloproteinase inhibitors, and have associated biosafety issues for human use. Furthermore these sources are only able to provide limited amounts of TIMP.

Stephen Quirk in U.S. Pat. No. 6,600,057 B2 titled Matrix metalloproteinase inhibitors has disclosed an invention which provides for compounds that are effective in treating disorders caused by the enzymatic activity of matrix metalloproteinases. These disorders include, but are not limited to, rheumatoid arthritis, osteoarthritis, periodontal disease, aberrant angiogenesis, tumor invasion and metastasis, corneal ulceration, and in complications of diabetes. The disclosed invention is also useful for treating wounds.

Nelson, Peter Jon, Ludwig-Maximilians-Universitat München in WO 2011063921 A1 titled Acceleration of wound healing by a tissue inhibitor of metalloproteinases (timp) linked to glycosylphosphatidylinositol (gpi)-anchors has disclosed an invention that relates to fusion constructs of glycosylphosphatidylinositol (GPI)-anchored tissue inhibitors of metalloproteinases (TIMPs) and their use for the treatment of cancer and in regenerative medicine. By this approach, the GPI-anchored TIMP proteins are incorporated into the surface membrane of cells. The fusion constructs are effective agents in accelerating wound healing.

It is an aspect of the present invention to overcome or at least alleviate one or more of the difficulties or deficiencies related to the prior art by providing a plentiful source of metalloproteinase and other protease inhibitor peptides from camelid serum/plasma and methods for purifying these inhibitor peptides from the camelid blood. Also it has been found that by inoculating camelids with purified human metalloproteinase enzyme and other antigens list given in claim 2 of similar molecular structure and adjuvant that specific homodimer antibodies generated in the inoculated camelid have the ability to inhibit metalloprotease enzymatic activity and other human proteases such as neutrophil elastase and this enhances the ability of the camelid serum to inhibit these enzymes for therapeutic application and increase supply of peptide inhibitors as therapeutics. Furthermore, compositions including inhibitors are disclosed as well as methods for treating various conditions and diseases using the naturally occurring metalloprotease inhibitory peptides and other human proteases in camelid serum or coupled with specific inhibitory homodimer antibodies generated by the camelid immune system following vaccination with specific human metalloprotease enzymes or antigens of similar structure. Described herein. These specific camelid inhibitory homodimer antibodies generated as a result of vaccination and the protease inhibitory peptides naturally present in camelid serum/plasma may be isolated for therapeutic use as outlined in this patent.

### SUMMARY OF THE INVENTION

The first aspect of the present invention provides a composition derived directly or indirectly from the blood/serum/Plasma of a camelid species, the composition comprising an inhibitor of a human metalloproteinase enzymes and human protease enzymes not requiring a metal group for activity. This invention has discovered that camelid serum/plasma contains effective inhibitory amounts of human metalloproteinase and general protease inhibitor peptides in concentration unlike other ruminant serum tested.

In a second aspect, the present invention provides a method for treating, preventing or ameliorating disorders associated with undesirable metalloproteinase enzyme or general protease enzyme activity, the method including administering to a human or animal in need thereof an effective amount of a composition comprising a metalloproteinase enzyme inhibitor peptide or a general protease enzyme inhibitor peptide present or generated homodimer antibody to a human protease enzyme capable of enzymatic inhibition or isolated from camelid serum/plasma. The methods are useful in the areas of enhancing chronic wound healing or general wound healing and for the enhanced healing of burns, disorders of skin or for the treatment of certain skin diseases and skin ageing, for the treatment of the gastrointestinal tract, and disorders of the cardiovascular system.

In a third aspect the present invention provides a method for at least partially purifying a human metalloproteinase enzyme or general protease enzyme inhibitor peptide or camelid homodimer antibody generated to inhibit human metalloproteinase enzyme or general protease, the method including the steps of one or more treatment steps for the camelid plasma selected from the group consisting of centrifugation, micro-filtration, ultra-filtration, ion-exchange chromatography, molecular sieve chromatography, affinity chromatography, reverse-phase high performance liquid chromatography and transient acidification.

### DETAILED DESCRIPTION OF THE INVENTION

In a first aspect the present invention provides a composition derived directly or indirectly from true camelid serum and or plasma the composition comprising a peptide inhibitor of a human metalloproteinase enzyme and or human protease enzymes. Applicants have demonstrated that naturally occurring serum peptides in camelid serum/plasma are useful as a source of human metalloproteinase enzyme inhibitors and of more general human protease enzyme inhibitors. The unexpected finding of these inhibitors in camelid serum/plasma provides a plentiful, renewable source of human metalloproteinase enzyme inhibitor peptides and non-metal human protease enzyme inhibitor peptides. As used herein the term “metalloproteinase” includes proteases that proteolytically degrade a component of the extracellular matrix. The term metalloproteinases includes but is not limited to (i) the collagenases (metalloproteinases-1, 8 and 13); (ii) the gelatinases A and B (metalloproteinase-2 and metalloproteinase-9); (iii) the stromelysins 1 and 2 (metalloproteinases-3 and 10); (iv) matrilysin (MMP-7); enamelysin (MMP-20), macrophage metalloelastase (MMP12), and MMP-19 (making up the classical metalloproteinases) and (v) the membrane-type metalloproteinases (MT-MMP-1 to 4 and stromelysin-3, MMP-11).

The present invention also includes the use of a peptide isolate and or formulation of camelid serum and or plasma containing these metalloprotease and there protease enzyme inhibitory peptides have not been found or isolated from other ruminant serum tested for example those other serum tested were bovine, caprine, equine species.

Preferably the peptide inhibitor present in the serum and or plasma of the camelid can inhibit human metalloproteinase enzyme and/of general non-metal requiring human protease enzyme are present at a concentration ranging from about 0.01 μg/ml to about 100 mg/ml in the formulation prepared for the application of this invention. More preferably the peptide inhibitor of human metalloproteinase and other proteases is present at a concentration ranging from about 0.1 μg/ml to about 1000 μg/ml. Even more preferably the camelid peptide inhibitor of human metalloproteinase and other proteases are present at a concentration ranging from about 1 μg/ml to 500 μg/ml. In a highly preferred embodiment, the protease inhibitor peptide from the camelid serum or plasma to human metalloproteinase or general non-metal containing protease is present at a concentration of about 11 μg/ml, or about 45 μg/ml or about 50 μg/ml as quantified by a fluorescence-quenching substrate assay.

In this invention the protease peptide inhibitor in the camelid plasma or serum is a tissue inhibitor of a metalloproteinase (TIMP). As used herein the term “tissue inhibitor of a metalloproteinase” includes but is not limited to polypeptides isolated from camelid blood which regulate the activity of human metalloproteinases which includes TIMP-1, TIMP-2, TIMP-3 and TIMP-4. The TIMP family is comprised of at least four distinct members (TIMP-1 to 4) which possess 12 conserved cysteine residues and express metalloproteinase inhibitory activity by forming non-covalent complexes with metalloproteinases. Specifically TIMPs bind to the highly conserved active zinc-binding site of the metalloproteinases in a 1:1 stoichiometry, but can also bind at other domains of metalloproteinase-2 and metalloproteinase-9.

Preferably the protease inhibitor camelid peptide is capable of inhibiting metalloproteinase 2 and/or metalloproteinase 9. Metalloproteinase 2 is also known as gelatinase A. Metalloproteinase 2 is a proteolytic enzyme having a molecular weight of 72 kDa which catalyses the degradation of collagen type IV by acting on the peptide bonds. Metalloproteinase 9 is also known as gelatinase B. Metalloproteinase 2 is a proteolytic enzyme having a molecular weight of 92 kDa which catalyses the degradation of collagen type IV by acting on the peptide bonds.

The protease peptide inhibitor isolated from camelid serum and or plasma is capable of inhibiting membrane type matrix metalloproteinases and non-metal bearing general protease enzymes.

Pharmaceutical and cosmetic compositions according to the present invention may be adapted for administration in any suitable manner. The composition may be adapted for internal or topical administration. The composition may be in an oral, injectable, topical or suppository form or formulated in a gel to make application to wound surfaces more convenient ocular. Preferred delivery routes include, dermal, intravaginal, intravenous, respiratory, and gastrointestinal delivery. It is to be understood that the compositions as described herein are not limited to use with humans, and include any animal that could benefit from the compositions of protease inhibitory peptides or homodimer antibodies with similar properties of protease inhibition.

Methods and pharmaceutical carriers for preparation of pharmaceutical compositions, including compositions for topical administration are well known in the art, as set out in textbooks such as Remington's Pharmaceutical Sciences, 18th Edition, Mack Publishing Company, Easton, Pa., USA.

Compositions of the present invention may be formulated so that they are suitable for oral administration. The compositions may be presented as discrete units such as capsules, sachets or tablets or in bandages each containing a predetermined amount of the active protease inhibitor peptide; as a powder or granules or gel, as a solution or a suspension in an aqueous or non-aqueous liquid; as a mouthwash or as an oil-in-water liquid emulsion or a water-in-oil liquid emulsion. The active ingredient may also be presented as a bolus, electuary or paste.

It should be understood that in addition to the ingredients particularly mentioned above, the compositions of this invention may include other agents conventional in the art having regard to the type of therapeutic in question, for example, those suitable for oral administration may include such further agents as sweeteners, thickeners and flavoring agents.

In a preferred form of the invention the compositions of the present invention include a carrier selected from the group consisting of a synthetic or biological polymer, glycosaminoglycan, or extracellular matrix molecule including fibrin, collagen, gelatin, a synthetic polymer, agarose, an alginate, methylcellulose, hyaluronic acid, a hydrocolloid, an alginate, saline solution, powder, ointment, salve or incorporated or impregnated into a dressing (absorbable and non-absorbable), a transdermal patch or releasable dressing associated with gauze, a bandage, suture, plaster, staple, prosthetic device, screw or plate (biodegradable or non-biodegradable), toothpaste, gum or resin for chewing, mouth wash or gel. The skilled artisan will be familiar with the appropriate carrier to use depending on the route or means for administration.

In another preferred form the composition has at least one further active ingredient selected from the group including antibiotics, anti-inflammatories, antiseptics, other growth promoter's e.g. insulin and anaesthetics. The compositions described herein may have other molecules associated therewith to aid releasability, stability, solubility, activity and/or association with wound healing, including carriers, solubilizing agents, and growth factors as discussed above.

In the above methods for treating wounds, camelid serum or plasma or isolates thereof or compositions of the present invention may be applied directly to wounds or in a biologically acceptable carrier to ensure sustained release at sufficient concentration in the wound environment. In treating a wound, the metalloprotease and non metal protease enzyme camelid peptide inhibitors may be associated with a wound support or gel. As used herein the term “wound support” includes any means which is used to support or secure a wound and includes a surgical securing means. The term includes plasters, dressings, sutures, staples and the like. The wound to be supported may be a wound created by surgery, or the result of accident or other injury. The camelid serum and or plasma or isolate thereof or composition may be present on the surface of the wound support or may be impregnated in the wound support/gel and is able to be released therefrom.

The wound to be treated according to this invention may be an ulcer caused by pressure, vascular disease, diabetes, autoimmune disease, sickle cell diseases or hemophilia; a result of surgery; therapeutically induced; associated with disorders of the central nervous system, and resulting from any exfoliative disease of the skin; a associated with either local or systemic infection or a corneal injury to the eye; a pathological wound; a traumatic or accidental wound; or a burn.

In a preferred method the concentration of the metalloproteinase or other protease enzyme inhibitor peptide or generated homodimer antibodies isolated or present in the camelid serum and or plasma is from about 0.1 ng/ml to about 10 μg/ml of fluid in the local environment at the wound site. Even more preferably the concentration of the camelid metalloproteinase or other protease enzyme peptide inhibitor present in the camelid serum and or plasma is from about 1 ng/ml to about 1 μg/ml of fluid in the local environment at the wound site.

The present invention also provides a method for preventing, ameliorating or treating a condition associated with a gastrointestinal injury, disease or ulcer, the method including administering to a human or animal in need thereof an effective amount of composition as described herein. In a preferred method the concentration of the camelid metalloproteinase/protease peptide inhibitor present in the medication should range from about 0.1 μg/ml to about 1000 μg/ml, even more preferably about 1 μg/ml to about 500 μg/ml.

As used herein the term “gastrointestinal injuries, diseases or ulcers” includes the following types of damage to or diseases of the gastrointestinal tract:
(a) dental and oral wounds, including those associated with periodontal disease;
(b) peptic ulceration of the duodenum, stomach or esophagus including gastric ulcers caused by radiation, non-steroidal anti-inflammatory drug (NSAID) therapy, helicobacter pylori bacteria or chemotherapy
(c) inflammatory bowel diseases such as ulcerative colitis or Crohn's disease;
(d) ulcers associated with stress conditions, for example burns, trauma, sepsis, shock, intracranial surgery or head surgery;
(e) damage to the lining of the alimentary tract, including mucositis or proctitis, resulting from radiotherapy and/or chemotherapy with agents such as mechlorethamine, melphalan, busulphan, cytarabine, floxuridine, 5-fluorouracil, mercaptopurine, methotrexate, thioguanine, bleomycin, actinomycin-D, daunorubicin, etoposide, mitomycin, vinblastine, vincristine, hydroxyurea or procarbazine;
(f) inadequate gut function or damage to the gut associated with prematurity such as necrotizing enterocolitis or poor gut motility;
(g) diarrhea conditions such as associated with bacterial, viral, fungal or protozoan infection;
(h) food intolerances such as coeliac disease;
(i) cancers of the gastrointestinal tract, including buccal cavity, oesophagus, stomach or bowel;
(j) surgically induced damage such as following partial gut resection, short gut syndrome, jejunostomy, ileostomy, colostomy;
(k) damage due to oesophageal reflux;
(l) conditions associated with loss of gut barrier function such as external burns, trauma, sepsis or shock;
(m) congenital conditions resulting in inadequate gastrointestinal function or damage such as volvulus and cystic fibrosis; and
(n) autoimmune diseases that affect the gut, such as Sjogren's Syndrome.

In the context of the invention, the term “effective amount” as used herein means an amount sufficient to elicit a statistically significant response at a 95% confidence level preferably, an effective amount is that amount to at least partially attain the desired response of a reduction in metalloproteinase and or other protease enzymatic activity at the disease tissue site.

The compositions may be administered in therapeutically effective amounts. A therapeutically effective amount means the amount required at least partly to attain the desired effect, ie to alleviate or prevent the symptoms of undesirable metalloproteinase/protease enzymatic activity in the wound or tissue or alternatively to delay the onset of, inhibit the progression of, or halt altogether, the onset or progression of the undesirable metalloproteinase/protease activity, or to reduce metalloproteinase/protease activity. Preferably the term “therapeutically effective amount” as used herein means amount sufficient to elicit a statistically significant response at a 95% confidence level.

In stent restenosis (ISR) peptides or homodimer antibodies, IV administration to prevent ISR after elevated MMP9. Protease inhibitor peptides and homodimer antibodies capable of protease inhibition can be delivered to patients such as ISR patients by:
(a) oral route loaded chitosan nanoparticles
(b) nasal route using absorption enhancers eg. Surfactants/bile salts/chitosan microsphere powder/carbopol 941/CMC
(c) vaginal route of delivery:—peptide inhibitors suspended in a poly (acrylic acid gel base)
(d) Ocular delivery:—peptide inhibitors suspended in absorption promoters eg. Bile salts, taurochlolic, deoxycholic acid
(e) Rectal route of delivery:—Utilize Erivative of peptide salicylats
(f) Targeted Loaded:—liposomes containing the MMP's inhibitory peptide of this patent

Such amounts will depend, of course, on the particular condition being treated, the severity of the condition, and individual patient parameters, including age, physical condition, size, weight and other concurrent treatment, and will be at the discretion of the attending physician. These factors are well known to those of ordinary skill in the art, and can be addressed with no more than routine experimentation. It is generally preferred that a minimum effective dose be determined according to sound medical judgment. It will be understood by those of ordinary skill in the art, however, that a higher dose may be administered for medical, psychological or other reasons.

Symptomatic patients may be identified after a careful history of the above symptoms in the injury, disease of ulcer and testing for metalloproteinase/protease enzymatic activity with a group of assays known in the art.

There are no limitations to the type of gastrointestinal injuries, diseases or ulcers that may be treated, and these include, but are not limited to dental and oral wounds, peptic ulcers, inflammatory bowel diseases, ulcers associated with stress conditions, damage caused by radiotherapy and/or chemotherapy, inadequate gut function or damage associated with prematurity, diarrhea conditions, damage caused by food intolerance, cancer of the gastrointestinal tract, surgically induced damage, damage caused by esophageal reflux, conditions associated with loss of gut barrier function, congenital conditions resulting in inadequate gastrointestinal function or damage, and autoimmune diseases that affect the gut.

The composition may be administered at any appropriate time including prior to, during or after the gastrointestinal injury, disease or ulcer has become evident.

The condition can be a dental or oral wound; peptic ulceration of the duodenum, stomach or esophagus; inflammatory bowel disease; an ulcer associated with stress conditions; damage to the lining of the alimentary tract; inadequate gut function or damage to the gut associated with prematurity; a diarrheal condition; a food intolerance; a cancer of the gastrointestinal tract; surgically induced damage to the gut; damage due to esophageal reflux; a condition associated with loss of gut barrier function; a congenital condition resulting in inadequate gastrointestinal function or damage; or an autoimmune disease that affects the gut.

In a fifth aspect the present invention is provided a method for preventing, ameliorating and/or treating disorders associated with undesirable metalloproteinase/protease enzymatic activity, the method including administering to a human in need thereof an effective amount of the protease inhibitory peptide present in the camelid serum and or plasma or the isolated peptide or antibody thereof or composition described herein. As used herein the term “disorders associated with metalloproteinase activity” includes the following:
(i) disorders of the cardiovascular system, where undesirable metalloproteinase activity has effected the remodeling of the cardiovascular system, including dilated cardiomyopathy, congestive heart failure, atherosclerosis, plaque rupture, reperfusion injury, ischemia, chronic obstructive pulmonary disease, angioplasty restenosis and aortic aneurism;
(ii) disorders of others tissues, where metalloproteinases are involved in the irregular remodeling including disorders of bone such as osteosclerosis or osteoporosis, disorders of other tissues such as liver cirrhosis and fibrotic lung disease, disorders of nervous tissues such as multiple sclerosis;
(iii) disorders relating to viral infection whereby metalloproteinase activity is altered, such as cytomegalovirus, retinitis, HIV and the resulting syndrome AIDS.
(iv) disorders relating to inflammation involving the implication of metalloproteinases such as inflammatory bowel disease, Crohn's disease, ulcerative colitis, pancreatitis, diverticulitis, asthma or related lung disease, rheumatoid arthritis, gout, Reiter's Syndrome, lupus erthmatosis, ankylosing spondylitis, autoimmune keratitis, pulmonary disease, bronchitis, emphysema, cystic fibrosis, acute respiratory distress syndrome;
(v) disorders relating to skin involving the implication of metalloproteinases, including psoriasis, scleroderma and atopic dermatitis or disorders relating to ultraviolet damage of skin which results in the skin having an aged and/or wrinkled appearance

Symptomatic patients are identified after a careful history of the above symptoms in tissue affected and testing for metalloproteinase activity with a group of assays available commercially.

In a preferred method the condition is a disorder of the cardiovascular system including but not limited to dilated cardiomyopathy, congestive heart failure, atherosclerosis, plaque rupture, reperfusion injury, ischemia, chronic obstructive pulmonary disease, angioplastly restenosis, aortic aneurism; a disorder of a tissue where a metalloproteinase is involved in the irregular remodeling including disorders of bone, liver, lung and nervous tissues; a disorder relating to viral infection whereby metalloproteinase activity is altered; a disorder relating to inflammation involving the implication of metalloproteinases; a disorder relating to skin involving the implication of a metalloproteinase, including but not limited to psoriasis, scleroderma and atopic dermatitis or disorders relating to ultraviolet damage of skin which results in the skin having an aged and/or wrinkled appearance. For all methods of treatment described herein the daily dosage can be routinely determined by the attending physician or veterinarian. Generally the dosage will vary according to the age, weight, and response of the individual patient, as well as the severity of the patient's symptoms. In general a suitable dose of the inhibitor of the invention will be in the range of about 0.1 μg to about 100 mg per kilogram body weight of the recipient per day, preferably in the range of about 1 μg to about 50 mg per kilogram body weight per day. However, the dose will also depend on the formulation and purity of the camelid serum and or plasma used and the conc. of inhibitory protease peptide present.

The present invention also provides a method for at least partially purifying or enriching a metalloproteinase enzyme inhibitor camelid peptide or antibody the method including the steps of isolating from the camelid serum/plasma thereof, and subjecting the camelid serum and or plasma to one or more treatment steps selected from the group consisting of centrifugation, micro-filtration, ultra-filtration, ion-exchange chromatography, molecular sieve chromatography, affinity chromatography, reverse-phase high performance liquid chromatography and transient acidification.

1. Sequence Description 1:

```
Lcu Lys Ala Mct Asp Pro Thr Pro Pro Lcu
                                    5 10
Trp Ilc Lys Thr Glu
                                  15
```

2. Collection of Sequences:

Phe-Leu-His = Peptide 1
Trp-Leu-Phe = Peptide 2
Trp-Leu-Try = Peptide 3
Trp-Leu-Arg = Peptide 4
Trp-Leu-His = Peptide 5
Phe-Leu-Phe = Peptide 6
Phe-Leu-Try = Peptide 7
Phe-Leu-Arg = Peptide 8

3. Collection of Sequences Coupled to Hydroxamate:

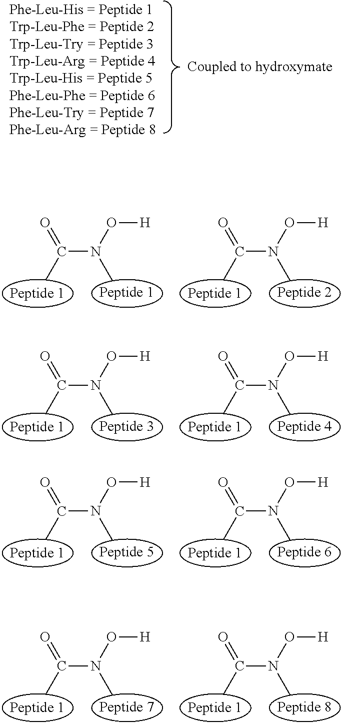

Any combination of the above peptides

1. A composition containing camelid serum or plasma filter sterilised containing endogenous protease enzyme inhibiting peptides of metalloproteinases and other proteolytic enzymes (e.g. elastase) for application to the chronic wound surface and/or burn surface to significantly enhance the wound healing process by inhibiting within a short timeframe the wound fluids proteolytic enzymes.

2. A composition in claim 1 containing camelid serum or plasma filter sterilized containing endogenous protease enzyme inhibiting peptides of metalloproteinases and other proteolytic enzymes (e.g. elastase) for application to the chronic wound surface and/or burn surface to cause inhibition of proteolytic enzymatic activity in the chronic wound fluid as demonstrated by proteolytic enzyme activity measurement using approved commercial kits provided such as WoundChek prior to camel plasma application and confirming proteolytic enzymatic activity has been inhibited in the wound fluid by the applied camel plasma after a short time period of a number of hours.

3. A composition in claim 2 of camelid serum and/or plasma which contains naturally a selection of proteases enzyme inhibitors including metalloprotease alone or combined with other active proteolytic peptide inhibitors derived from the serum or plasma of anyone or a combination of opossum, mongoose, meerkats, wood rats or cotton rat which when applied to the surface of chronic wounds and/or burns significantly enhances the healing process versus current standard methods of care by inhibiting proteolytic enzymatic activity in the chronic wound fluid this serum/plasma can be demonstrated to work by measuring the wound fluid protease enzymatic activity using a commercially available wound fluid protease assay kit both before camelid serum or plasma application and at a time points a number of hours following application to confirm complete inhibition of wound fluid proteolytic enzymatic activity. This new wound fluid environment generated by the inhibition of wound fluid Proteases Enzymes, when maintained with continual monitoring to ensure that protease activity is absent will allow normal healing to progress to wound closure.

4. A composition according to claim 1 wherein the camelid serum and/or plasma is supplemented with bio active insulin to further enhance efficacy of wound healing in chronic diabetic ulcer treatment.

5. A composition according to claim 1 or claim 2 wherein the camelid serum and/or plasma is supplemented with other known prescription agents approved for wound healing.

6. A composition according to claims 1, 2 and 3. Where in the camelid serum and/or plasma or other defined animal serum or plasma is formulated in a gel for ease of application to the wound surface. These gels can be chosen from either hydrogel and/or alginate formulations which are currently available for wound dressing formulations.

7. A composition according to any one of claims 1 to 4 wherein the protease peptide inhibitors including metalloprotease enzyme inhibitor peptides are isolated from camelid blood/serum/plasma or the other defined animals listed in claim 3 for use in the confirmed inhibition of chronic wound fluid proteolytic enzymatic activity utilising the commercially available wound fluid protease activity assay kits.

8. A composition according to any one of claims 1 to 5 wherein the peptide inhibitor of a protease enzymes is isolated from the camelid serum/plasma/to a purity of from about 70% to about 99% with respect to the total protein content of the isolate and utilised in a formulation to inhibit proteolytic enzymatic activity in chronic wound fluid.

9. A method for treating, preventing or ameliorating a disorder associated with undesirable metalloproteinase or protease enzyme activity in chronic wound fluid by incorporation of the dried plasma and/or isolated protease inhibitory peptides outlined in claims 1 to 4 above into the body of a dressing or bandage.

10. A method according to claims 1 to 7 wherein the disorder is a wound caused by pressure, vascular disease, diabetes, autoimmune disease, sickle cell diseases or haemophilia; a result of surgery; therapeutically drug or radiation induced or associated with disorders of the central nervous system or neoplasm and/or resulting from any exfoliative disease of the skin; or a corneal injury or disease of the eye.

11. A method according to claims 1 to 8 wherein the protease inhibitor plasma or isolated peptides are administered by IV, enterically coated or specially formulated for buccal or anal route delivery where the disorder is a dental or oral wound; mucositis; peptic ulceration of the duodenum, stomach or esophagus; inflammatory bowel disease; proctitus; an ulcer associated with stress conditions; damage to the lining of the alimentary tract; inadequate gut function or damage to the gut associated with prematurity; a diarrhoea condition; a food intolerance; a cancer of the gastrointestinal tract; surgically induced damage to the gut; damage due to oesophageal reflux; a condition associated with loss of gut barrier function; a congenital condition resulting in inadequate gastrointestinal function or damage; or an autoimmune disease that affects the gut.

12. A method according to claims 1 to 11 wherein the disorder is in the cardiovascular system selected from the group including dilated cardiomyopathy, congestive heart failure, atherosclerosis, plaque rupture, reperfusion injury, ischemia, chronic obstructive pulmonary disease, angioplastly restenosis, in-stent restenosis, aortic aneurism; a disorder of a tissue where a metalloproteinase is involved in the irregular remodelling including disorders of bone, liver, lung and nervous tissues; a disorder relating to viral infection whereby metalloproteinase activity or other protease is elevated ; a disorder relating to inflammation involving the presence of elevated metalloproteinases or elastase; a disorder relating to skin involving the enhanced activity of metalloproteinase or other protease enzymes needing to be inhibited by non-toxic topical administration of the products of this patent including but not limited to psoriasis, scleroderma and atopic dermatitis, improve scar tissue healing in skin graft or cosmetic surgery procedures or disorders relating to ultraviolet damage of skin which results in the skin having an aged and/or wrinkled appearance due to enhanced metalloprotease or other protease activity which can be inhibited by the peptides either in the animal plasma as outlined in claim 3 or their isolated or synthesis peptides.

13. A method for at least partially purifying or enriching the metalloproteinase or other protease inhibitor peptides from the defined animals in claim 3's blood/serum or plasma, the method including the steps selected from the group consisting of centrifugation, micro-filtration, ultra-filtration, ion-exchange chromatography, molecular sieve chromatography, affinity chromatography, reverse-phase high performance liquid chromatography and transient acidification.

14. A method according to claims 1 to 13 wherein the disorder is resultant from elevated human neutrophil elastase and/or plasma kallikrein and these proteolytic enzymes are inhibited by therapeutic application of the camel peptides and generated homodimer antibodies of this patent.

15. A method of producing the camelid protease inhibitory peptides and homodimer antibodies of this patent by inoculating camelids with purified snake venoms and adjuvant.

16. A method of producing the camelid protease inhibitory peptides and homodimer antibodies of this patent by inoculating camelids with isolated and/or recombinant human metalloprotease enzymes alone and/or combined with isolated and/or recombinant human neutrophil elastase enzyme

17. That by inoculating camelids with purified human metalloproteinase enzyme and other antigens of similar molecular mimic structure and adjuvant that specific homodimer antibodies are generated in the inoculated camelid serum and plasma having the ability to inhibit metalloprotease enzymatic activity and this enhances the ability of the camelid serum to inhibit these enzymes for therapeutic application and increase supply of inhibitors.

18. A composition containing any combination of the amino acid sequences listed in this patent, peptide 1 to 8 for application to the chronic wound surface and/or burn surface to significantly enhance the wound healing process by inhibiting within a short timeframe the wound fluids proteolytic enzymes.

19. A composition containing any combination of the amino acid sequences listed in this patent, peptide 1 to 8 coupled to Hydroxamate for application to the chronic wound surface and/or burn surface to significantly enhance the wound healing process by inhibiting within a short timeframe the wound fluids proteolytic enzymes.

20. A composition according to claims 1, 2, 15, 16, 18 and 19 in combination formulation as a medication in the treatment of elevated metalloproteinases and other proteolytic enzymes in a disease state.

21. A composition wherein the inhibitor is a tissue inhibitor of a metalloproteinase (TIMP).

22. A composition wherein the TIMP is a TIMP-2 polypeptide or a functional equivalent or fragment thereof.

23. A composition according to claim 22 wherein the TIMP-2 has a molecular weight of about 21,000 Da as determined by SDS-PAGE and/or has an isoelectric point of about 7.0.

24. A composition according to claim 23 wherein the TIMP-2 comprises the following N-terminal sequence: NH2-CSCSPVHP.

25. A composition according to claim 24 wherein the TIMP-2 comprises the following sequence:

26. NH2_CSCSPVHPQQAFCNADIVIRAKAVNKKEVDSGNDIYGNPIKRIQYEI KQIKMFKGPDQDIEFIYTAPAAAVCGVSLDIGGKKEYLIAGKAEGNGNMHI TLCDFIVPWDTLSATQKKSLNHRYQMGCECKITRCPMIPCYISSPDECLW MDWVTEKNINGHQAKFFACIKRSDGSCAWYRGAAPPKQEFLDIEDP_C OOH, or a functional equivalent or fragment thereof.

27. A method for treating, preventing or ameliorating a disorder associated with undesirable metalloproteinase activity, the method including administering to an animal in need thereof an effective amount of a composition according to any one of claims 22 to 25.

28. A method according to claim 26 wherein the disorder is a wound caused by pressure, vascular disease, diabetes, autoimmune disease, sickle cell diseases or hemophilia; a result of surgery; therapeutically induced;
associated with disorders of the central nervous system, and resulting from any exfoliative disease of the skin; a associated with either local or systemic infection such as yaws, HIV, chicken pox or herpes infection; congenital; a corneal injury to the eye; a pathological wound; a traumatic or accidental wound; or a burn.

29. A method according to claim 26 wherein the disorder is a dental or oral wound; peptic ulceration of the duodenum, stomach or esophagus; inflammatory bowel disease; an ulcer associated with stress conditions; damage to the lining of the alimentary tract; inadequate gut function or damage to the gut associated with prematurity; a diarrheal condition; a food intolerance; a cancer of the gastrointestinal tract; surgically induced damage to the gut; damage due to esophageal reflux; a condition associated with loss of gut barrier function; a congenital condition resulting in inadequate gastrointestinal function or damage; or an autoimmune disease that affects the gut.

30. A method according to claim 26 wherein the disorder is a disorder of the cardiovascular system selected from the group including dilated cardiomyopathy, congestive heart failure, atherosclerosis, plaque rupture, reperfusion injury, ischemia, chronic obstructive pulmonary disease, angioplastly restenosis, aortic aneurism; a disorder of a tissue where a metalloproteinase is involved in the irregular remodeling including disorders of bone, liver, lung and nervous tissues; a disorder relating to viral infection whereby metalloproteinase activity is altered; a disorder relating to inflammation involving the implication of metalloproteinases; a disorder relating to skin involving the implication of a metalloproteinase, including but not limited to psoriasis, scleroderma and atopic dermatitis or disorders relating to ultraviolet damage of skin which results in the skin having an aged and/or wrinkled appearance.
